(19) United States
(12) Patent Application Publication
(10) Pub. No.: US 2013/0252202 A1
(43) Pub. Date: Sep. 26, 2013

(54) METHOD FOR PLANNING A DENTAL PROSTHESIS IMPLANT ARRANGEMENT AND A REFERENCE ARRANGEMENT

(71) Applicants: Roland Pardeller, Terlan (IT); Andreas Geier, Tisens (IT)

(72) Inventors: Roland Pardeller, Terlan (IT); Andreas Geier, Tisens (IT)

(21) Appl. No.: 13/828,874

(22) Filed: Mar. 14, 2013

Related U.S. Application Data
(63) Continuation-in-part of application No. PCT/EP2012/054943, filed on Mar. 21, 2012.

(51) Int. Cl. A61C 8/00; A61C 1/08; A61B 6/14; A61C 13/00; A61B 6/03

(52) U.S. Cl. CPC A61C 8/0089; A61C 13/0004; A61B 6/032; A61B 6/14; A61C 1/088; A61C 8/0001
USPC 433/173

(57) ABSTRACT

The invention includes an apparatus and a method for planning a dental prosthesis implant arrangement. An oral reference arrangement (e.g., a dental tray) can include an alignment portion, configured for extra-oral placement, and having surfaces configured to receive and display an optical reference plane projected from an external reference system. The optical reference plane can provide a reference to align the oral reference arrangement and/or teeth and a jaw of a patient for imaging and subsequent planning of a dental prosthesis implant. A method of planning a dental prosthesis implant arrangement can include initial imaging of the teeth and the jaw of the patient, while the above-described oral reference arrangement is detachably mounted to the teeth and/or jaw of the patient. Three-dimensional information relating to the oral reference arrangement, the teeth, and/or the jaw can be used to generate a virtual model of the teeth and/or the jaw.

### RELATED APPLICATIONS

This patent document is a continuation-in-part, and claims the benefit of priority of, PCT Patent Application No. PCT/EP2012/054943, filed on Mar. 21, 2012, which claims priority to German Patent Application No. DE 10 2011 014555.9, filed on Mar. 21, 2011, the entirety of each of the disclosures of the afore-mentioned patent documents is explicitly incorporated by reference herein.

### TECHNICAL FIELD

The present invention relates to an apparatus and a method for planning a dental prosthesis implant arrangement.

### BACKGROUND

Dental prosthesis implant arrangements can be planned by an actual model of a patient's teeth. The actual model can be made with the aid of a dental impression. The actual model reproduces, to a large extent, exactly the relative positions of the teeth to one another (so-called “occlusion”) as well as the topography of the surrounding gum. A preliminary planning of existing dental prosthesis implants is subsequently performed with the help of a wax model (so-called “wax-up”).

The wax model provides a first impression as to how the missing natural teeth of the patient can be replaced by one or more dental prosthesis implants. The wax model serves as a template for preparing a further model from a radiopaque material, which provides a sufficiently good contrast in X-ray images. The model from radiopaque material can be introduced into the patient and, if required, adapted to the patient. With the model inserted, a computed tomography (CT) scan or a dental volume tomography (DVT) scan can be made of the jaw of the patient. The data obtained thereby can provide information as to how the model lies in relation to the teeth and the jaw. On the basis of the data, a subsequent planning of the implant positions and, in particular, of the boreholes to be drilled to anchor the implant or implants can be carried out with the aid of an implant planning program.

At least two sessions with the patient are required for the method described above because the “wax-up” and the production of the model from radiopaque material require a considerable investment of time, which furthermore can only be carried out by specialized personal and thus incurs commensurate costs. In addition, a correct positioning of the model from radiopaque material in the oral cavity of the patient and even a postionally exact alignment of the patient during the CT or DVT scan is often a problem. In the present art, the radiographic measurements need to be transformed into the appropriate coordinate system. Such transformations of the 3-dimensional measurements result in computational effort on the one hand, but, more importantly, in a loss of accuracy with a given measurement data resolution. To achieve the same accuracy in the resolution of the data, data collection must be enhanced, resulting in higher radiologic burden on the patient, greater measurement time and cost.

Subsequent changes to the model also entail considerable time and expense. In the event of the patient deciding that the overall impression produced by the model is not satisfactory, the wax-up procedure and the production of the radiopaque model have to be repeated.

A further disadvantage of the previously described method is that a decision, as to whether the surgical procedure for implanting the dental prosthesis implant arrangement can actually be carried out as planned, can be made only after the CT or DVT scan has been performed. This results from the fact that the condition of the jaw bone is only established after said scan. Under certain circumstances, the model has to be reworked, which can require one or more of the aforementioned steps to be repeated.

Altogether, it can be concluded that the method previously described is associated with a considerable expenditure of time and money.

### OVERVIEW

The present inventors recognize, among other things, that a method and related arrangements facilitating a faster and more cost effective planning of dental prosthesis implants is needed. The present disclosure now provides an oral reference arrangement having an extra-oral alignment portion that is referenced to an external, known coordinate system. The extra-oral alignment portion allows the measurement of the patient's oral cavity to be spatially oriented to the known coordinate system, thus requiring less, if any, spatial transformation of the intra-oral measurement, allowing the radiologic measurements to be taken with a lower resolution to reach a certain accuracy and reducing the radiologic burden on the patient. Moreover, the extra-oral alignment portion is captured with high resolution optical scans, thus enabling a verification and consolidation of the alignment, and providing high resolution correction data for the intra-oral radiographic data, if required.

The present disclosure relates to an apparatus, namely an oral reference arrangement, and a method of using an oral reference arrangement to plan a dental prosthesis implant arrangement. The reference arrangement can be configured such that a portion is placed in a patient's oral cavity, while another portion can be retained extra-orally. An external reference system having a known coordinate system can be associated with the oral reference arrangement to capture data regarding the reference arrangement, one or more teeth, a jaw, and/or certain external facial features of the patient. The captured data can be used to generate a virtual model for use in planning a dental prosthesis implant arrangement. The reference arrangement can facilitate a fixed spatial orientation of the teeth and the jaw of the patient, in relation to an external reference system, such that three-dimensional data regarding the teeth and the jaw can be captured and used to create a virtual model for planning dental prosthesis implant arrangements.

In one embodiment, the reference arrangement can include a connecting unit configured to be detachably secured on a patient, particularly to the patient's teeth or to auxiliary points that are connected to the patient's jaw in a spatially fixed manner. Such auxiliary points can, for example, be temporary auxiliary implants for securing the reference arrangement. Attaching the reference arrangement to the teeth, the jaw and/or to the auxiliary points can ensure that the reference arrangement, the teeth, and the jaw are in a fixed spatial relationship to one another.

The reference arrangement can also include an alignment unit disposed for extra-oral placement. The spatial position of the reference arrangement can be subsequently aligned by aligning the alignment unit relative to an external reference system, specifically, an optical reference image or plane, which is associated with the known coordinate system of the external reference system, that is projected onto one or more surfaces of the alignment unit. In other words, the reference arrangement is aligned to the external reference system through an optical adjustment method so that the teeth and the jaw of the patient can thereby be brought into a defined position in relation to the external reference system.

After alignment of the reference arrangement, the teeth and the jaw of the patient can be simultaneously and three-dimensionally captured and a corresponding data set is generated to more precisely determine the orientation of the reference arrangement relative to the external reference system.

The optical adjustment method to align an extra-oral alignment unit, includes a combination of an optical reference plane associated with an external reference system and a high resolution optical imaging method to capture data regarding the spatial position of the extra-oral alignment unit. An optical adjustment method ultimately reduces computational effort, provides greater accuracy in processing imaging data regarding the oral cavity, and eliminates the need to enhance the resolution of the radiographic data collection, thereby reducing the radiographic burden on the patient.

Because the extra-oral alignment portion is optically aligned first, the jaws and teeth are in a known position with respect to the imaging apparatus and external reference system at the time of data capture. Thus, this process requires less correction in processing the captured data (i.e. data is collected in the right coordinate system), which allows generation of highly precise data using lower resolution radiologic data collection because measured data points do not need to be transferred to a different coordinate system, which in turn lowers the radiologic burden.

A corresponding data set can be generated from the teeth and/or the jaw scan and a virtual model of the teeth and/or the jaw is produced on the basis of the data set. The prior alignment of the reference arrangement with respect to an external reference system can result in creation of a virtual model that is unambiguously oriented in the space and can, therefore, be used for the further planning of a dental prosthesis implant arrangement.

Data reflecting the anatomical characteristics in the region of the teeth and the jaw can be obtained by simultaneous data capture of the teeth and the jaw. Any suitable method can be used to capture data, for example an imaging method. The captured data comprise a data set which forms the basis for a virtual model of the teeth and/or the jaw. The virtual model is, for example, calculated from the generated data set.

The virtual model can be generated directly from the generated data set. Hence, there are no burdensome intermediate steps required between the data acquisition and the creation of the virtual model, except for transformations of the data set, e.g., transformations of the data format. Consequently, the data set can be fed substantially directly into the model calculation.

In contrast to conventional methods, a three-dimensional data capture of the current situation of the patient's teeth and the jaw takes place at the beginning of the planning process. The data set obtained from three-dimensional data capture can be clearly defined due to the prior alignment of the reference arrangement and the attachment thereof to the teeth, the jaw, and/or to the corresponding auxiliary points, simplifying the subsequent steps in the planning process.

The term “dental prosthesis implant arrangement” includes individual dental prosthesis implants, implant arrangements and combinations of a plurality of individual dental prosthesis implants.

According to an embodiment of the method, an extra-oral region of the patient, in particular a section of the face of the patient in the region of the jaw can be three-dimensionally captured to take into account aspects of the extra-oral aesthetics when planning the dental prosthesis implant arrangement.

In other embodiments of the method, three-dimensional data capture of reference elements associated with the reference arrangement take place simultaneously with the three-dimensional data capture of the extra-oral region of the patient. Alternatively or additionally, simultaneous three-dimensional data capture of the teeth and the jaw comprise simultaneous three-dimensional data capture of reference elements of the reference arrangement. In some embodiments, the reference elements are placed on the extra-oral alignment unit for three-dimensional data capture of the reference elements in combination with the teeth and/or jaw rand an extra-oral region of the patient.

According to the embodiments of the method previously described, it is therefore possible for the teeth, the jaw, the extra-oral region of the patient, and/or reference elements of the reference arrangement to be simultaneously and three-dimensionally captured , such as through imaging, in order to be able to generate a data set for a specific application.

According to one embodiment of the method, a reference arrangement is used, in which at least one reference element, preferably a plurality of reference elements, is disposed on the extra-oral alignment unit of the reference arrangement. Preferably the at least one reference element is configured so that it can be optically captured, including by a high resolution optical imaging device.

In other embodiments of a reference arrangement, reference elements can comprise a spherical reference body connected to a shaft, the shaft being configured for detachable connection to the reference arrangement. Such reference elements can be optically captured to facilitate an exact referencing of the captured data of the patient. A detachable connection allows reference elements to be secured to the reference as needed or in different positions for specific applications, and allows reuse of reference elements when replacing the reference arrangement.

High resolution optical imaging of the extra-oral alignment unit, in combination with extra-orally disposed reference elements and/or extra-oral portions of the patient in the jaw region allow for consolidation and verification of the reference arrangement alignment, and thus, alignment of the patient's teeth and jaw, to the optical reference plane associated with the external reference system. It should be noted that because extra-oral imaging data will inherently have greater resolution that intra-oral imaging data, an additional benefit of the present optical alignment method, and its focus on alignment of an extra-oral portion of the reference arrangement, is that accurate alignment and data capture can occur with reduced radiologic burden on the patient.

In one embodiment of the method, the above-described virtual model can be used in a computer assisted virtual preliminary planning of the dental prosthesis implant arrangement. For example, the virtual model produced from the data set can be fed into a suitable planning program. In the event that reference elements are provided on the reference arrangement, the spatial position of the reference elements can facilitate an assignment of the data points of the captured data set on the basis of the nominal position of the reference elements, which is or can be deposited as a reference system in a dental prosthesis implant planning program. In other words, reference elements can create an unambiguous fixed reference system, to which the data points can be “mounted”. Data sets collected with other imaging methods can be integrated in a simple manner on the basis of fixed reference system.

With the aid of the program, the form and position of the dental prosthesis implant arrangement can be planned, adjusted and optimized on the basis of the virtual model. The virtual model can be used directly in preliminary planning, eliminating the need for intermediate steps are otherwise required to use a virtual model for preliminary planning In other words, the virtual model is designed to directly serve as a basis for the preliminary planning.

In addition, a virtual dental prosthesis implant model and/or a virtual model of the oral cavity of the patient can be developed within the framework of the virtual preliminary planning, to serve as a basis for further planning steps.

The virtual dental prosthesis implant model and/or the virtual model of the oral cavity of the patient (or a model of a part of the oral cavity), can be presented to the patient relatively quickly after completion of the three-dimensional data capture of the teeth and the jaw. Thus, the preliminary planning, and substantial parts of the planning of the dental prosthesis implant arrangement, can be carried out during one session. Changes to the virtual dental prosthesis implant model and/or the virtual model of the oral cavity of the patient can be easily performed on the model and can be immediately visualized. In addition, an estimate of costs can be made on the basis of the data. Therefore, all information essential for making a decision can be made immediately available to the patient.

Data sets of the teeth and the jaw can, for example, be combined with data sets of the extra-oral jaw region of the patient to take into account the impact of the virtually ascertained dental implant arrangement on the extra-oral aesthetics of the patient.

After approval of the dental prosthesis implant model and/or the virtual model of the oral cavity, at least a portion of the components of the dental prosthesis implant arrangement can be machined using the data of the virtual dental prosthesis implant model. The electronically available data of the dental prosthesis implant model can be directly provided to suitable machines or devices to produce the dental prosthesis implant components without loss of time or precision. In this context, the term “directly” includes pure transformations of the data set, such as a transformation of the data format.

In one embodiment of the method, the data capture of the teeth, the jaw, and the reference elements of the reference arrangement can take place with the aid of a computed tomography (CT) scan or a dental volume tomography (DVT) scan to provide an exact image of the present situation of the teeth and the jaw of the patient. The corresponding data set can be quickly processed and edited.

The data capture of the extra-oral region of the patient and/or reference elements disposed on extra-oral portion of the reference arrangement, including the extra-oral alignment unit, can be performed optically, such as with a high resolution optical CT scan or a high resolution optical DVT scan.

In an embodiment, at least one referencing component associated with an external reference system, such as an optical reference image or an optical reference plane, can be projected onto the reference arrangement by means of a beam of light. In all embodiments of the methods disclosed herein, it is understood that for properly alignment of the reference arrangement, the alignment unit must first be adjusted such that an optical reference plane is properly projected onto at least one reference surface of the alignment unit, after which the reference arrangement is aligned with respect to the optical reference plane so projected onto the reference surface. The alignment of the reference arrangement can be oriented relative to the optical reference image or plane. When so aligned, the reference arrangement is disposed in a predefined position relative to the optical reference image or plane. The position of the optical reference image or plane is known and clearly defined in relation to the gantry of a CT or DVT scanner. Use of an optical reference image or plane that is associated with an external reference system can allow for an accurate and easy orientation of the reference arrangement, and can reduce computational efforts to generate accurate three-dimensional data of the patient's anatomy. Furthermore, because the present method does not require a mechanical positioning assembly, the area scanned can be free of structures that could potentially perturb the scan process or data generated from the scan. The acquisition and combination of intra-oral and extra-oral data (i.e., data capture of the connecting unit and the alignment unit) can be simplified by using the optical orientation approach of the above-described methods. In many cases, radiological exposure of the patient can be reduced because the present methods optimize data precision and consolidation.

In an embodiment, the reference arrangement can be brought into a substantially horizontal position in alignment with an optical reference plane. Alignment of the reference arrangement within the optical reference plane and/or parallel thereto can, for example, be achieved by a distinctive point in the head region of the patient being aligned relative to a further marking, such as a laser marking.

In this context, the term “beam of light” means a beam of arbitrary geometry. For example, the beam of light can have a circular, oval or rectangular cross-section, or be fanned out by suitable means (e.g., by a moving mirror), in order to generate the desired optical reference plane. An extensive light beam, which can be used as a reference plane, can also be generated by an aperture arrangement.

In yet another embodiment of the method, the reference arrangement is detachably secured to the teeth and/or, directly or indirectly, to the jaw of the patient by an impression compound. The invention further relates to an oral reference arrangement for use in a dental prosthesis implant planning The reference arrangement can comprise a connecting unit for establishing a detachable connection of the reference arrangement to the patient's teeth and/or to auxiliary points that are connected to the patient's jaw in a spatially fixed manner. The reference arrangement can include an alignment unit, which is integral with the connecting unit, and is disposed extra-orally such that the reference arrangement can be selectively adjusted relative to an external reference system. In an embodiment, the connecting unit has at least one surface that is essentially or generally coplanar with at least one surface of the alignment unit. For example, a top surface of the connecting unit and a top surface of the alignment unit are essentially or generally coplanar, and/or a bottom surface of the connecting unit is essentially or generally coplanar with a bottom surface of the alignment unit. The coplanar features of the connecting unit and the alignment unit permit alignment of the plane of the alignment unit (e.g., to an external reference system) to be translated to corresponding alignment of the connecting unit and, thus, the teeth and jaw of the patient.

In alternate embodiments, the reference arrangement can include at least one reference element, and preferably a plurality of reference elements, each of which are disposed at a predetermined position with on the reference arrangement. The reference elements constitute a fixed reference system that can be coupled to an external reference system through the alignment unit. The patient's teeth and/or jaw can be brought into a fixed spatial reference by means of the detachable connection of the connecting unit to the patient. The reference arrangement allows alignment of the teeth and/or the jaw of a patient to an external reference system to facilitate three-dimensional capture of the teeth and the jaw in a known spatial position. The corresponding data can be used in further treatment steps and/or for planning a dental prosthesis implant arrangement. The alignment unit provides a means by which the reference arrangement can be brought into the desired position and the position of the reference arrangement can be verified in relation to an external reference system.

In another embodiment, at least some reference elements are disposed on the connecting unit. In an alternate embodiment, all reference elements can be located on the connecting unit. The reference elements can be at least partially intra-orally disposed when attached to the connecting unit. The spatial proximity of the reference elements to the teeth or to the jaw can facilitate an exact determination of the position of the individual components of the teeth or the jaw relative to the fixed reference system defined by reference elements.

In one embodiment, the alignment unit can comprise at least one reference surface configured to align the reference arrangement relative to an external reference system. In one embodiment, the alignment unit can comprises at least two reference surfaces. In an alternate embodiment the at least two reference surfaces are angled relative to each another, and are configured to align the reference arrangement relative to at least one reference plane associated with the external reference system. The at least two reference surfaces can enclose, for example, an angle between 0° and 180°, non-inclusive, preferably an angle between approximately 90° and 180°, non-inclusive, and most preferably between 120° and 150°, inclusive. In still other embodiments, the at least one reference plane can comprise at least two reference planes, each one of the at least two reference planes being projected on one of the at least two reference surfaces. Disposition of a first reference surface in a medial-lateral direction, and disposition of a second reference surface at an angle between the medial-lateral direction and the anterior-posterior direction allows an optical reference plane from a single optical source to be projected in an anterior-posterior direction and be simultaneously received and displayed upon both the first and second reference surfaces.

The alignment unit can have a horizontally extending, cantilevered configuration to facilitate adjustment of the lateral tilt of the reference arrangement, and, thus, the tilt of the patient's head/teeth/jaw relative to an external reference system, in particular, an optical reference plane associated with an external reference system as described herein.

A cantilevered configuration of alignment unit having at least one reference surface angled with respect to the anterior-posterior direction, facilitates adjustment of the “nodding” tilt (up/down) of the patient's head with respect to the optical reference plane.

The reference surfaces can be disposed substantially vertical or substantially perpendicular in relation to a plane formed by the alignment unit. In one embodiment, the reference surfaces are configured such that at least one reference plane can be optically projected on the reference surfaces, and in turn, the optical reference plane can be displayed or optically “read on” the reference surfaces to facilitate an alignment of the reference arrangement relative to the optical reference plane. In alternate embodiments, the reference surfaces are configured so that the optical reference plane can be easily recognized. For example, the reference surface can be roughened, textured or patterned to aid visualization and/or display of the optical reference plane on the reference surfaces.

A reference plane can, for example, be easily projected by means of a laser. The reference plane generated by the laser can be used for aligning a reference feature that is provided on the reference surface. The position of the reference arrangement is, for example, adjusted until the optical reference plane and the reference feature are aligned or are disposed parallel to each other. A reference feature can include a line or other indicia, a notch, a groove, a ridge, or visible feature, including an edge of the reference surface.

In another embodiment, the alignment unit can be in the shape of a grip or handle to facilitate placement, manipulation and alignment of the reference arrangement. In an alternate embodiment, the alignment unit defines a plane extending substantially horizontal in relation to the head. The reference surfaces can be disposed be on the periphery of the alignment unit, preferably the reference surfaces are disposed opposite the patient's face.

To facilitate an alignment of the reference arrangement with respect to a sagittal plane of the patient, the alignment unit can have a reference feature, for example a recess, a notch, a groove, a ridge or other visible feature, disposed on the a side, edge or surface of a reference arrangement, including a side, edge or surface of a reference surface. In one embodiment, the reference feature can extend in the anterior-posterior direction, i.e., from the face toward the back of the head. In one embodiment, the reference feature can be used to align the reference arrangement with a facial feature, for example the front teeth, to align the reference arrangement in the sagittal plane.

A compact design of the reference arrangement can be achieved if the alignment unit and the connecting unit are disposed substantially in one plane (i.e., are coplanar) and/or have substantially the same thickness as measured from their respective top surfaces to their respective bottom surfaces.

An embodiment of the reference arrangement can include detachable attachment to the teeth and/or jaws using an impression compound. The connecting unit can be configured with a receiving area for receiving and retaining the impression compound. The receiving area can comprise an indentation in a top surface of the connecting unit. The receiving area can further comprise, at least in certain sections, a top edge delimitation.

An embodiment of the top edge delimitation can include, at least in certain sections, an undercut to prevent separation of the impression compound from the connecting unit when it is being detached from the teeth and/or jaw of the patient. When the impression compounds cures, a positive connection can be established between the impression compound and the top edge delimitation.

The receiving area can additionally or alternatively include a plurality of retention channels, which traverse the receiving area of the connecting unit from a top surface or upper (attachment) side of the connecting unit (including a retaining area lower surface) to a bottom surface of the connecting unit. The retention channel is at least open at the top surface. In an embodiment, retention channel widens as the channel extends from the top surface towards the bottom surface. In yet another embodiment, the retention channel has a conical shape, the cone widening as the channel extends from a top surface to a bottom surface of the connecting unit. When the impression compound cures, a positive fit or connection is produced by impression compound filling the retention channels. A positive fit prevents the impression compound from detaching from the reference arrangement when the apparatus is removed from the mouth of the patient.

In other embodiments, the receiving area can be furnished with spacers that extend upwardly from the receiving area lower surface. In an alternative embodiment, spacers are arranged in a web-like manner to prevent an extensive contact between parts of the teeth and the lower surface of the receiving area.

Reference elements can be removably attached to the reference arrangement. Removable attachment allows reuse of said reference elements and positioning of the reference elements at different locations on the reference arrangement, for example, to implement different fixed reference systems.

In an embodiment, a reference element can comprise a spherical reference body connected to a shaft that is configured to be introduced into a borehole on the reference arrangement. The spherical configuration of the reference body makes it possible to determine a precise position of the reference element. At least a portion of the reference element can comprise a partially radiopaque material. Preferably, at least the reference body is radiopaque to facilitate determination of the positioning of the reference element during CT or DVT imaging.

In one embodiment of the reference arrangement, the connecting unit and the alignment unit are integrally formed, allowing for simple and cost effective production. In particular, the reference arrangement can be made from plastic, such as Plexiglas. The reference arrangement can be configured for single use.

To further illustrate the oral reference arrangement and related method disclosed herein, a non-limiting list of embodiments is provided here:

In Embodiment 1, an oral reference arrangement can comprise a connecting unit, configured for detachable connection to a patient's teeth or to one or more auxiliary attachment points on an interior jaw, defining an orientation plane, and an alignment unit fixedly attached to, or coextensive with, the connecting unit, and including at least one plane that is generally coplanar with the orientation plane. The alignment unit can include a at least one reference surface arranged for extra-oral placement and configured to receive and display an optical reference plane projectable by an external reference system.

In Embodiment 2, the reference arrangement of Embodiment 1 can optionally be configured such that the at least one reference surface comprises a first reference surface and a second reference surface.

In Embodiment 3, the reference arrangement of Embodiments 1 or 2 can optionally be configured such that the at least one of a top surface or a bottom surface of the alignment unit is coplanar with the orientation plane.

In Embodiment 4, the reference arrangement of Embodiment 1 can be optionally configured to further comprise a plurality of reference elements, each of which is configured to be attached to a predetermined position on the connecting unit or the alignment unit.

In Embodiment 5, the reference arrangement of Embodiment 1 can optionally be configured such that the at least one reference surface defines a plane that is substantially vertical or perpendicular to a top surface of the alignment unit.

In Embodiment 6, the reference arrangement of any one or any combination of Embodiments 1-5 can optionally be configured such that the at least one reference surface includes a roughened, textured, or patterned portion to facilitate display of the optical reference plane.

In Embodiment 7, the reference arrangement of any one or any combination of Embodiments 2-6 can optionally be configured such that the first and second reference surfaces are disposed at an angle to each other.

In Embodiment 8, the reference arrangement of Embodiment 6 can optionally be configured such that the angle is between 0° and 180°, non-inclusive.

In Embodiment 9, the reference arrangement of Embodiments 7 or 8 can optionally be configured such that the angle is between 90° and 180°, non-inclusive.

In Embodiment 10, the reference arrangement of Embodiments 7-9 can optionally be configured such that the angle is between about 120° and about 150°, inclusive.

In Embodiment 11, the reference arrangement of any one or any combination of Embodiments 1-10 can optionally be configured such that at least one of the connecting unit or the alignment unit is selectively adjustable relative to the optical reference plane.

In Embodiment 12, the reference arrangement of any one or any combination of Embodiments 1-11 can optionally be configured such that the at least one reference surface defines a plane perpendicular to an anterior-posterior direction.

In Embodiment 13, the reference arrangement of any one or any combination of Embodiments 1-12 can optionally be configured such that the at least one reference surface defines a plane that is angled with respect to an anterior-posterior direction.

In Embodiment 14, the reference arrangement of any one or any combination of Embodiments 2-13—can optionally be configured such that the first reference surface is laterally adjoined, directly or indirectly, to the second reference surface.

In Embodiment 15, the reference arrangement of any one or any combination of Embodiments 2-14 can optionally be configured such that the first reference surface defines a plane perpendicular to an anterior-posterior direction, and the second reference surface defines a plane disposed at an angle to the first reference surface, between the anterior-posterior direction and a medial-lateral direction.

In Embodiment 16, the reference arrangement of any one or any combination of Embodiments 1-15 can optionally be configured such that the alignment unit includes a marking, a notch, a groove, or a ridge extending substantially in an anterior-posterior direction and disposed on at least one of a top surface or a bottom surface, opposite the top surface.

In Embodiment 17, the reference arrangement of any one or any combination of Embodiments 1-16 can optionally be configured such that the alignment unit is shaped to form a grip or a handle.

In Embodiment 18, the reference arrangement of any one or any combination of Embodiments 1-17 can optionally be configured such that the alignment unit and the connecting unit have substantially an identical thickness, as measured from each unit's top surface to each unit's bottom surface, opposite the top surface.

In Embodiment 19, the reference arrangement of any one or any combination of Embodiments 4-18 can optionally be configured such that the plurality of reference elements are removably attached to the connecting unit or the alignment unit and selectively adjustable relative to the optical reference plane.

In Embodiment 20, the reference arrangement of any one or any combination of Embodiments 4-19 can optionally be configured such that each one of the plurality of reference elements comprises a spherical body portion connected to a shaft, with the shaft configured to be inserted into a borehole disposed within a surface of the connecting unit or a surface of the alignment unit.

In Embodiment 21, the reference arrangement of any one or any combination of Embodiments 4-20 can optionally be configured such that certain ones of the plurality of reference elements are removably attached to the connecting unit and other ones of the plurality of reference elements are removably attached to the alignment unit.

In Embodiment 22, a method for planning a dental prosthesis implant can comprise providing or obtaining a reference arrangement including an oral portion and an alignment portion, and detachably securing the oral portion in an oral cavity of a patient while orienting the alignment portion extra-orally of the patient. The method can further comprise projecting a beam of light, associated with an external reference system, onto the alignment portion, including defining an optical reference plane, and aligning a spatial position of the reference arrangement relative to the optical reference plane. Further yet, the method can comprise the steps of imaging, contemporaneously, the one or more teeth, and a jaw of the patient, generating a data set including three-dimensional information from the imaging, and creating a virtual model of the teeth or the jaw based, at least in part, on the data set.

In Embodiment 23, the method of Embodiment 22 can optionally be modified such that the step of aligning includes high resolution optical scanning of the alignment unit.

In Embodiment 24, the method of Embodiment 22 or 23 can optionally be modified such that the step of aligning includes orienting the alignment portion relative to the optical reference plane.

In Embodiment 25, the method of any one or any combination of Embodiments 22-24 can optionally be modified such that the step of aligning includes aligning a boundary, an edge, a surface, a marking, or a reference feature of the alignment portion parallel to the optical reference plane.

In Embodiment 26, the method of any one or any combination of Embodiments 22-25 can optionally be modified such that the alignment portion is configured with at least one reference surface, and the step of projecting optionally includes projecting the optical reference plane onto the at least one reference surface.

In Embodiment 27, the method of any one or any combination of Embodiments 22-26 can optionally be modified such that the at least on reference surface comprises a first reference surface and a second reference surface, and the step of projecting optionally includes projecting the optical reference plane onto the first reference surface and the second reference surface.

In Embodiment 28, the method of Embodiment 27 can optionally be modified such that the step of projecting optionally includes projecting the optical reference plane from a single light source simultaneously onto the first reference surface and the second reference surface.

In Embodiment 29, the method of any one or any combination of Embodiments 22-28 can optionally be modified such that the step of imaging includes imaging an extra-oral jaw region of the patient.

In Embodiment 30, the method of Embodiment 29 can optionally be modified such that the reference arrangement comprises a plurality of reference elements removably attached to the alignment portion, and the step of imaging includes obtaining three-dimensional optical data capture of the plurality of reference elements contemporaneously with three-dimensional optical data capture of the extra-oral region of the patient.

In Embodiment 31, the method of Embodiment 30 can optionally be modified such that the step of imaging includes contemporaneous three-dimensional optical data capture of the teeth, the jaw, and the plurality of reference elements.

In Embodiment 32, the method of any one or any combination of Embodiments 22-31 can optionally be modified to further include the step of performing a computer-assisted virtual preliminary planning of the dental prosthesis using the virtual model.

In Embodiment 33, the method of Embodiment 32 can optionally be modified such that the step of creating the virtual model includes creating a virtual model within a framework of the computer-assisted virtual preliminary planning, in which the anticipated costs for a production of an actual dental prosthesis implant are calculated and displayed.

In Embodiment 34, the method of Embodiment 33 can optionally be modified to further include the step of machining at least a portion of the actual dental prosthesis implant using the data associated with the virtual model.

In Embodiment 35, the method of any one or any combination of Embodiments 22-34 can optionally be modified such that the step of imaging includes using a computed tomography (CT) scan or a dental volume tomography (DVT) scan.

In Embodiment 36, the method of any one or any combination of Embodiments 22-35 can optionally be modified such that the step of detachably securing includes placing an impression compound between the oral portion and the teeth or the jaw of the patient.

In Embodiment 37, the method of any one or any combination of Embodiments 22-36 can optionally modified such that the reference arrangement comprises any one or any combination of the oral reference arrangement of Embodiments 1-21.

In Embodiment 38, the oral reference arrangement and related method of any one (or portion of any one) or any combination of Embodiments 1-37 can optionally be configured such that all elements or options recited are available to use or select from.

These and other examples and features of the present oral reference arrangement and method will be set forth in part in the following Detailed Description, drawings and claims. This Overview is intended to provide non-limiting examples of the present subject matter—it is not intended to provide an exclusive or exhaustive explanation. The Detailed Description below is included to provide further information about the present oral reference arrangement and related method.

### BRIEF DESCRIPTION OF THE DRAWINGS

In the following, the present disclosure will be explained with respect to embodiments and with reference to the accompanying drawings. In the drawings, like numerals can be used to describe similar elements throughout the several views. Like numerals having different letter suffixes can be used to represent different views or features of similar elements. The drawings illustrate generally, by way of example, but not by way of limitation, various embodiments discussed in the present patent document.

FIG. 1 illustrates a perspective view of an embodiment of a reference arrangement.

FIG. 2 illustrates a top view of an embodiment of a reference arrangement.

FIGS. 3 illustrates a front view, or anterior-to-posterior view, of an embodiment of a reference arrangement.

FIG. 4 illustrates a side view, or lateral view, of an embodiment of a reference arrangement.

FIG. 5 illustrates an embodiment of a reference element.

### DETAILED DESCRIPTION

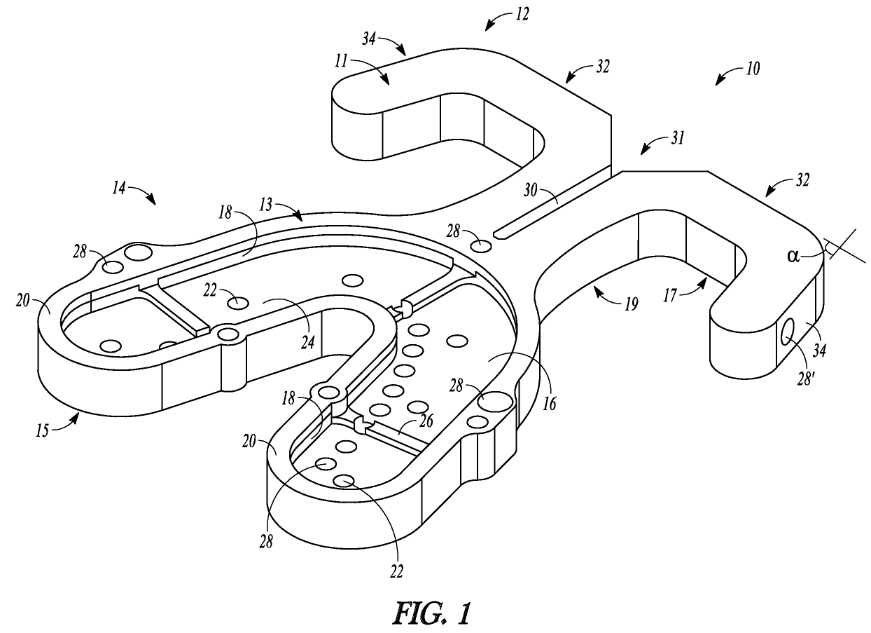

FIG. 1 illustrates an embodiment of an oral reference arrangement 10 comprising an alignment unit 12 and a connecting unit 14. The alignment unit 12 can be an extra-oral portion used to align a spatial position of the reference arrangement 10, relative to an external reference system, when the reference arrangement 10 is secured to teeth and/or a jaw of a patient. A bridge portion 19 can be disposed between the connecting unit 14 and alignment unit 12 to provide a transition between intra-oral and extra-oral portions of the reference arrangement 10.

A receiving area 16, formed as an indentation in the connecting unit 14, can be configured to receive an impression compound into which the teeth and/or the jaw of the patient are pressed. In one embodiment, the reference arrangement 10 can be used on an upper jaw or on a lower jaw of the patient.

After the reference arrangement 10 is fitted to the patient, the impression compound is allowed to cure so that the reference arrangement 10 is connected to the patient in a spatially fixed manner. A head of the patient can be subsequently aligned relative to an external reference system, as is explained in greater detail below.

When the impression compound has cured, the impression compound can be simultaneously connected to the reference arrangement 10, because the compound has penetrated into one or more undercuts 18 in a top edge delimitation 20, which laterally delimits the receiving area 16, before it has cured. In a further embodiment, the receiving area 16 can include one or more retention channels 22, which traverse the connecting unit 14 from a receiving area lower surface 24 to a bottom surface 15 of the connecting unit. The retention channels 22 are open at least at the receiving area lower surface so that the impression compound can penetrate the one or more channels. In an alternate embodiment, the retention channels 22 widen as they extend from the receiving area lower surface 24 toward the bottom surface 15 of the connecting unit 14. In yet another embodiment, the retention channels 22 widen in a conical configuration. The retention channels 22 can act in a similar fashion as the one or more undercuts 18.

To accommodate the limited space in the oral cavity of the patient, in certain embodiments, the connecting unit 14 can have a flattened and/or narrower configuration in a retromolar region, for example, the top edge delimitation 20 can be lowered.

To prevent the patient's teeth from resting on a large portion of the receiving area lower surface 24 when the reference arrangement 10 is secured to the patient, in some embodiments, the receiving area 16 can include spacers 26, which extend upwardly from the receiving area lower surface 24.

In yet another embodiment, the connecting unit 14 can include a plurality of reference element boreholes 28 configured to receive reference elements. When reference elements are inserted into the boreholes 28, the reference elements define a fixed reference system. A fixed reference system does not require that all boreholes 28 are equipped with reference elements. By selectively equipping specified boreholes 28 with reference elements, different fixed reference systems can be defined. The boreholes 28 can pierce the connecting unit 14 so that the reference elements can be inserted into the connecting unit 14 from the top surface 13, 24 as well as from the bottom surface 15 of the connecting unit 14.

In one embodiment, in the usage position, the extra-oral alignment unit 12 of the reference arrangement 10 can extend generally horizontally with respect to the head of the patient. In an embodiment, the alignment unit 12 can include a symmetrical form with respect to a reference feature, such as a line, a notch, a groove, a ridge or other reference feature or reference marking 30. In one embodiment, a reference feature 30 can extend substantially in an anterior-posterior direction. When positioning the reference arrangement 10, the reference feature 30, such as a notch, can be aligned such that it runs approximately between the front teeth of the patient, defining a sagittal plane passing from front (anterior) to back (posterior) direction. As can be seen in FIG. 3, the reference feature 30 can also be provided on the bottom edge 38 of the reference arrangement 10, which is not visible in FIG. 1. A V-shaped recess 31 adjacent the reference feature 30 can facilitate the correct positioning of the reference arrangement 10 with respect to the sagittal plane.

The alignment unit can include at least one reference surface 32, 34. In alternate embodiments the alignment unit comprises at least a first reference surface and a second reference surface 32, 34. After securing the reference arrangement 10, the head of the patient can be aligned by at least one optical reference plane R that is associated with an external reference system. In alternate embodiments, the at least one optical reference plane can comprise a first optical reference plane R1 and a second optical reference plane R2. In one embodiment the first optical reference plane and the second optical reference plane can be generated from a single optical reference source, for example a single beam of light. In yet another embodiment, a first reference plane R1 can be projected from a first beam of light, and a second optical reference plane R2 can be projected from a second beam of light, the first and second beams of light can be generated by a single source (simultaneously or in series) or by separate sources (simultaneously or in series).

In one embodiment, the optical reference plane R can be projected onto the at least one reference surface 32, 34 of the alignment unit 12. The at least one reference surface 32, 34 can be substantially planar. In another embodiment, as shown in FIG. 3, when viewed from a frontal view, the at least one reference surface 32 can define a planar surface that extends substantially horizontally, in the medial-lateral direction. In alternate embodiment the at least one reference surface 32 can extend perpendicularly with respect to one or more of an anterior-posterior direction or the reference feature 30. In another embodiment, the at least one reference surface 34 can extend at an angle with respect to one or more of an anterior-posterior direction, a medial-lateral direction, or a reference feature 30. In still another embodiment, the at least one reference surfaces includes a first reference surface 32 extending medial-laterally, in a plane substantially perpendicular to the anterior-posterior direction, and a second reference surface 34 extending in a plane that is disposed at an angle between an anterior-posterior direction and a medial-lateral direction.

In yet another embodiment, an optical reference plane R can be simultaneously projected onto a first reference surface 32 and a second reference surface 34. In one embodiment, the first reference surface 32 extends in a plane that is perpendicular to at least one of the anterior-posterior direction or a reference feature 30, and a second reference surface 34 that extends in a plane that is disposed angled at an angle with respect to the medial-lateral direction least one of an anterior-posterior direction, a reference feature 30 and/or the first reference surface 32. The reference surfaces 32, 34 can be disposed substantially perpendicular with respect to a top surface 11 of the alignment unit 12. The reference surfaces 32, 34 can enclose an angle α, between 0° and 180°, preferably an angle which is between 90° and 180°. Most preferably, the angle is between approximately 120° and 150°. In one embodiment, at least two reference surfaces 32, 34 can be angled in relation to each other to permit two-dimensional orientation and alignment with respect to one or more optical reference planes R, R1, R2. Disposition of a first reference surface 32 in a medial-lateral direction, and disposition of a second reference surface 34 at an angle between the medial-lateral direction and the anterior-posterior direction allows an optical reference plane R from a single optical source to be projected in an anterior-posterior direction and be simultaneously received and displayed upon both the first and second reference surfaces 32, 34.

The alignment unit 12 can have a horizontally extending, cantilevered configuration to facilitate adjustment of the lateral tilt of the reference arrangement 10 and, thus, the tilt of the patient's head/teeth/jaw relative to an external reference system, in particular, an optical reference plane R associated with an external reference system as described herein. A cantilevered configuration of alignment unit 12 having at least one reference surface 34 angled with respect to the anterior-posterior direction, facilitates adjustment of the “nodding” tilt (up/down) of the patient's head with respect to the optical reference plane R.

If a projection of the optical reference plane R onto the reference surfaces 32, 34 extends parallel to the respective upper edge 36 and lower edge 38 of the reference surfaces 32, 34, the reference arrangement 10 is oriented in the reference plane R. An alignment with the reference plane R does not have to be oriented to an edge of the reference surfaces 32, 34. Reference feature 30 in the form of lines or other indicia, notches, grooves, ridges can, alternatively or additionally, be provided on the reference surface faces 32, 34 to facilitate alignment between the optical reference plane R and the reference surfaces 32, 34.

The alignment unit 12 can allow for an exact alignment of the reference arrangement 10 with respect to the optical reference plane R. Because the alignment section 12 can be integral with the connecting unit 14, positioning errors due to improper assembly can be diminished. The reference arrangement 10 can, for example, be a cost effective plastic part that is designed as a disposable item. Alternatively, acrylic glass is a suitable material for manufacturing the reference arrangement 10.

In an embodiment, the alignment unit can include boreholes 28′ configured for receipt and attachment of reference elements 40.

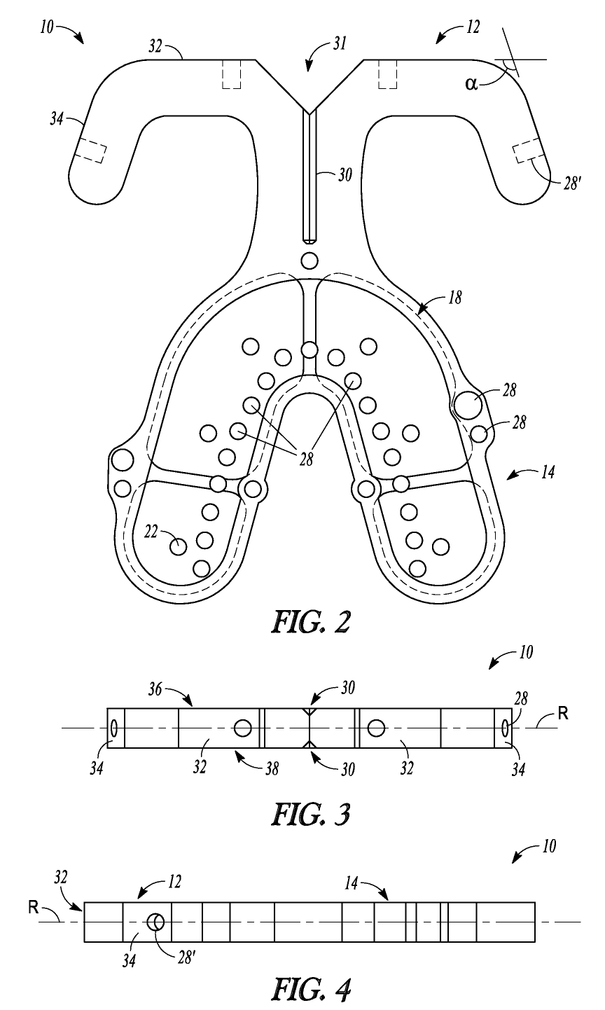

In FIG. 2, an embodiment of the reference arrangement 10 illustrates the relative position of reference surfaces 32, 34. In the depicted exemplary embodiment, the angle α is slightly greater than 105°. The angle can be varied as circumstances require.

It can be important for an unambiguous a first alignment of the reference arrangement 10 with respect to the reference plane R (e.g., alignment of lateral tilt) that a first reference surface 32 is not parallel to a second reference surface 34. It can be important for an unambiguous second alignment of the reference arrangement 10 with respect to the reference plane R (e.g. alignment of a “nodding tilt” or up/down) that a second reference surface 34 is not perpendicular to a first reference surface 32 and is not parallel to an anterior-posterior projected optical reference plane R.

In FIG. 2, the contours of the undercuts 18 are indicated by dashed lines in the region of the connecting unit 14. The dashed lines in the alignment unit 12 indicate the position of the boreholes 28′.

FIG. 3 illustrates a front view of the reference arrangement 10. In an aligned state of the reference arrangement 10, the reference plane R runs parallel to an upper edge 36, as well as parallel to a lower edge 38 of the reference surfaces 32, 34.

It can further be seen in FIG. 3 that the reference arrangement 10 can be of flat design, i.e., the alignment unit 12 and the connecting 14 are coplanar with each other and with the reference plane R. Alignment unit 12 and the connecting unit 14 have a substantially identical thickness, as seen in FIG. 4.

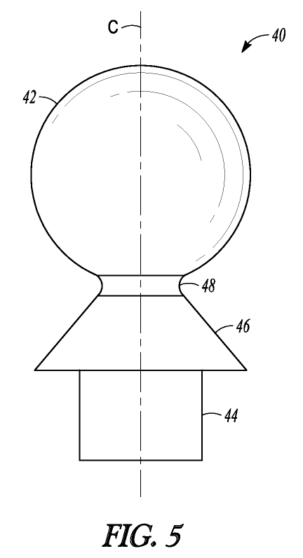

FIG. 5 illustrates a reference element 40, which has a spherical reference body 42 and a shaft 44. In order to secure the reference element 40 to the reference arrangement 10, the shaft 44 is inserted into a borehole 28, 28′. A truncated, cone-shaped intermediate section 46 of the reference element 40 defines how far the shaft 44 can be inserted into the borehole 28, 28′, enabling the position of the center C of the reference body 42 to be clearly defined with respect to the reference arrangement 10. The shape of the intermediate section 42 of the reference body 42 can lead to a constriction 48 so that the spherical shape of the reference body 42 is easy to recognize in a CT or DVT scan. As a result, the position of the center C of the reference body 42 can also be exactly determined. The positions of the reference bodies 42 ascertained by the CT or DVT scans can also, for example, be compared to corresponding nominal values in order to validate the data obtained by the scans and, if necessary, to correct the data. In addition, the reference elements 40 constitute a fixed reference system, which makes it possible to couple data sets obtained by different methods.

Based on electronically available data from the scans, the teeth and the jaw of the patient can be exactly captured with the help of suitable programs, and the position of the teeth and the jaw can be depicted precisely. In one embodiment, the dental prosthesis implants can also be virtually planned with great precision using suitable planning software. In addition, a preview of the teeth and the jaw can be created which shows the teeth and the jaw after implantation of the planned dental prosthesis implants. The computer assisted data acquisition of the current state of the oral cavity of the patient, the virtual preliminary planning of the dental prosthesis implant arrangement on the basis of the acquired data, and the display of a preview of the situation after completed treatment can be carried out easily and quickly in comparison to conventional methods. A virtual model of the teeth after a successful treatment can be presented to the patient in the first session. If needed, the model can also be virtually adapted with minimal cost and effort. An estimate of costs can also be quickly developed on the basis of the existing data. As soon as the patient agrees with the proposed implantation model, a more precise planning can be started on the basis of the existing data, which planning, for example, comprises a more precise virtual definition of the boreholes to be drilled for the placement of the implants. The existing data can also be used to produce implants specific to the particular patient.

It can be seen from the above embodiments that a physical model of the dental prosthesis arrangement does not have to be developed in the planning phase of the present method, because the impression compound only secures the reference arrangement to the teeth or the jaw of the patient so that the patient's head can be correctly aligned by the reference arrangement. The ability to dispense with a physical model can reduce the procedural steps and the costs of treatment. In addition, an impression as to how a patient's teeth will look upon completion of treatment can be conveyed to the patient more quickly than in conventional dental prosthesis planning methods.

Nevertheless, existing data can be used to produce such a physical model. A physical model can also be developed using the impression of the patient's teeth in the impression compound.

In the event of inconsistent usages between this document and any document incorporated by reference herein, the usage in this document controls.

In this document, the terms “a” or “an” are used to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated.

In this document, “dental prosthesis implant arrangement” refers to individual dental prosthesis implants, as well as arrangements and combinations of a plurality of individual dental prosthesis implants. “Optical reference plane” refers to any image, or group of images, that can be projected onto a reference surface and that facilitates alignment of the reference arrangement relative to an external reference system, including, without limitation, a plane, a straight line, other geometrical shape.

In this document, and if used, “anterior” refers to a direction generally toward the front of a patient, “posterior” refers to a direction generally toward the back of the patient, “medial” refers to a direction generally toward the middle of the patient, and “lateral” refers to a direction generally toward the side of the patient. In this document, the phrase “anterior/posterior direction” is used to include an anterior to posterior direction or a posterior to anterior direction.

In the appended claims, the terms “having,” “including” and “in which” are used as the plain-English equivalents of the respective terms “comprising” and “wherein.” The terms “having”, “including” and “comprising” are open-ended, that is, an apparatus, system, kit, or method that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above Detailed Description includes references to the accompanying drawings, which form a part of the Detailed Description. The drawings show, by way of illustration, specific embodiments in which oral reference arrangement assembly and related methods can be practiced. These embodiments are also referred to herein as “examples.”

The above Detailed Description is intended to be illustrative, and not restrictive. For example, the above-described examples (or one or more elements thereof) can be used in combination with each other. Other embodiments can be used, such as by one of ordinary skill in the art upon reviewing the above description. Also, various features or elements can be grouped together. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

The Abstract is provided to allow the reader to quickly ascertain the nature of the technical disclosure. It is submitted with the understanding that it will not be used to interpret or limit the scope or meaning of the claims.

### REFERENCE NUMERALS

10 reference arrangement
11 alignment unit top surface
12 alignment unit
13 connecting unit top surface
14 connecting unit
15 connecting unit bottom surface
16 receiving area
17 alignment unit bottom surface
18 undercut
19 bridge portion
20 top edge delimitation
22 retention opening
24 receiving area lower surface
26 spacer
28, 28′ reference element borehole
30 reference feature
31 recess
32 anterior surface
34 lateral surface
36 top edge
38 bottom edge
40 reference element
42 reference body
44 shaft
46 intermediate section
48 constriction
α angle
C center line of reference element
R reference plane

What is claimed is:

1. An oral reference arrangement, comprising:
a connecting unit, configured for detachable connection to a patient's teeth or to one or more auxiliary attachment points on an interior jaw, defining at least one orientation plane;
an alignment unit fixedly attached to, or coextensive with, the connecting unit, and including at least one plane that is generally coplanar with the orientation plane,
the alignment unit further including at least one reference surface arranged for extra-oral placement and configured to receive and display an optical reference plane projectable by an external reference system.

2. The reference arrangement of claim 1, wherein the at least one reference surface comprises a first reference surface and a second reference surface.

3. The reference arrangement of claim 1, wherein at least one of a top surface or a bottom surface of the alignment unit is coplanar with the orientation plane.

4. The reference arrangement of claim 1, further comprising a plurality of reference elements, each of which is configured to be attached to a predetermined position on the connecting unit or the alignment unit.

5. The reference arrangement of claim 1, wherein the at least one reference surface defines a plane that is substantially vertical or perpendicular to a top surface of the alignment unit.

6. The reference arrangement of claim 1, wherein the at least on reference surface includes a roughened, textured, or patterned portion to facilitate display of the optical reference plane.

7. The reference arrangement of claim 2, wherein the first and second reference surfaces are disposed at an angle to each other.

8. The reference arrangement of claim 7, wherein the angle is between 0° and 180°, non-inclusive.

9. The reference arrangement of claim 8, wherein the angle is between 90° and 180°, non-inclusive.

10. The reference arrangement of claim 9, wherein the angle is between about 120° and about 150°, inclusive.

11. The reference arrangement of claim 1, wherein at least one of the connecting unit, or the alignment unit is configured to be selectively adjustable relative to the optical reference plane.

12. The reference arrangement of claim 1, wherein the at least one reference surface defines a plane perpendicular to an anterior-posterior direction.

13. The reference arrangement of claim 1, wherein the at least one reference surface defines a plane that is angled with respect to an anterior-posterior direction.

14. The reference arrangement of claim 2, wherein the first reference surface is laterally adjoined, directly or indirectly, to the second reference surface.

15. The reference arrangement of claim 2, wherein the first reference surface defines a plane perpendicular to an anterior-posterior direction, and the second reference surface defines a plane disposed at an angle to the first reference surface, between the anterior-posterior direction and a medial-lateral direction.

16. The reference arrangement of claim 1, wherein the alignment unit includes a marking, a notch, a groove, or a ridge extending substantially in an anterior-posterior direction and disposed on at least one of a top surface or a bottom surface, opposite the top surface.

17. The reference arrangement of claim 1, wherein the alignment unit is shaped to form a grip or a handle.

18. The reference arrangement of claim 1, wherein the alignment unit and the connecting unit have substantially an identical thickness, as measured from each unit's top surface to each unit's bottom surface, opposite the top surface.

19. The reference arrangement of claim 4, wherein the plurality of reference elements are configured to be removably attached to the connecting unit or the alignment unit and selectively adjustable relative to the optical reference plane.

20. The reference arrangement of claim 4, wherein each one of the plurality of reference elements comprises a spherical body portion connected to a shaft, the shaft configured to be inserted into a borehole disposed within a surface of the connecting unit or a surface of the alignment unit.

21. The reference arrangement of claim 4, wherein certain ones of the plurality of reference elements are removably attached to the connecting unit and other ones of the plurality of reference elements are removably attached to the alignment unit.

22. A method for planning a dental prosthesis implant, comprising:
providing or obtaining a reference arrangement including an oral portion and an alignment portion;
detachably securing the oral portion in an oral cavity of a patient while orienting the alignment portion extra-orally of the patient;
projecting a beam of light, associated with an external reference system, onto the alignment portion, including defining an optical reference plane;
aligning a spatial position of the reference arrangement relative to the optical reference plane;
imaging, contemporaneously, one or more teeth, and a jaw of the patient;
generating a data set including three-dimensional information from the imaging; and
creating a virtual model of the teeth or the jaw based, at least in part, on the data set.

23. The method of claim 22 , wherein the step of aligning includes high resolution optical scanning of the alignment unit.

24. The method of claim 22, wherein the step of aligning includes orienting the alignment portion relative to the optical reference plane.

25. The method of claim 22, wherein the step of aligning includes aligning a boundary, an edge, a surface, a marking, or a reference feature of the alignment portion parallel to the optical reference plane.

26. The method of claim 22, wherein the alignment portion comprises at least one reference surface, and the step of projecting optionally includes projecting the optical reference plane onto the at least one reference surface.

27. The method of claim 26, wherein the at least one reference surface comprises a first reference surface and a second reference surface, and the step of projecting includes projecting the optical reference plane onto the first reference surface and the second reference surface.

28. The method of Embodiment 27, wherein the step of projecting comprises projecting the optical reference plane from a single light source simultaneously onto the first reference surface and the second reference surface.

29. The method of claim 22, wherein the step of imaging includes imaging an extra-oral jaw region of the patient.

30. The method of claim 22, wherein the reference arrangement further comprises a plurality of reference elements removably attached to the alignment portion, and wherein the step of imaging includes obtaining three-dimensional optical data capture of the plurality of reference elements contemporaneously with three-dimensional optical data capture of the extra-oral region of the patient.

31. The method of claim 30, wherein the step of imaging includes contemporaneous three-dimensional optical data capture of the teeth, the jaw, and the plurality of reference elements.

32. The method of claim 22, further comprising the step of performing a computer-assisted virtual preliminary planning of the dental prosthesis using the virtual model.

33. The method of claim 32, wherein the step of creating the virtual model includes creating a virtual model within a framework of the computer-assisted virtual preliminary planning, and wherein anticipated costs for a production of an actual dental prosthesis implant are calculated and displayed.

34. The method of claim 33, further comprising the step of machining at least a portion of the actual dental prosthesis implant using the data associated with the virtual model.

35. The method of claim 22, wherein the step of imaging includes using a computed tomography (CT) scan or a dental volume tomography (DVT) scan.

36. The method of claim 22, wherein the step of detachably securing includes placing an impression compound between the oral portion and the teeth or the jaw of the patient.

37. The method of claim 22, wherein the reference arrangement comprises the reference arrangement of claim 1.
